(19) United States
(12) Patent Application Publication
(10) Pub. No.: US 2005/0033362 A1
(43) Pub. Date: Feb. 10, 2005

(54) High strength suture with collagen fibers

(76) Inventor: R. Donald Grafton, Naples, FL (US)

Correspondence Address: DICKSTEIN SHAPIRO MORIN & OSHINSKY LLP, 2101 L Street, NW, Washington, DC, 20037, US

(21) Appl. No.: 10/912,221

(22) Filed: Aug. 6, 2004

Related U.S. Application Data
(63) Continuation-in-part of application No. 10/358,399, filed on Feb. 5, 2003.
Application No. 10/358,399 is a continuation-in-part of application No. 10/160,176, filed on Jun. 4, 2002.
Application No. 10/160,176 is a continuation-in-part of application No. 09/950,598, filed on Sep. 13, 2001, now Pat. No. 6,716,234.
(60) Provisional application No. 60/330,913, filed on Nov. 2, 2001.
(60) Provisional application No. 60/350,040, filed on Jan. 23, 2002.
(60) Provisional application No. 60/354,499, filed on Feb. 8, 2002.
(60) Provisional application No. 60/492,972, filed on Aug. 7, 2003.

(51) Int. Cl. A61L 17/00

(52) U.S. Cl. 606/228

(57) ABSTRACT

A high strength abrasion resistant surgical suture material with improved tie down characteristics and enhanced tissue remodeling and biocompatibility includes protein polymer fibers blended with synthetic fibers. The suture features a multifilament cover formed of strands of ultra high molecular weight long chain polyethylene braided with polyester, nylon or other nonabsorbable materials, along with fibers of a protein polymer, such as collagen. The cover surrounds a core formed of twisted strands of ultrahigh molecular weight polyethylene. The core also can include collagen fibers. The suture, provided in a #2 size, has the strength of #5 Ethibond, is ideally suited for most orthopedic procedures, and can be attached to a suture anchor or a curved needle. The collagen fiber enhances tissue remodeling into the suture strand, thus improving biocompatibility.

### CROSS-REFERENCE TO RELATED APPLICATIONS

This application is a continuation-in-part of U.S. application Ser. No. 10/358,399, filed Feb. 5, 2003, which is a continuation-in-part of U.S. application Ser. No. 10/160,176, filed Jun. 4, 2002, which claims the benefit of U.S. provisional application Ser. No. 60/330,913, filed Nov. 2, 2001, U.S. provisional application Ser. No. 60/350,040, filed Jan. 23, 2002, and U.S. provisional application Ser. No. 60/354,499, filed Feb. 8, 2002, and which is a continuation-in-part of U.S. application Ser. No. 09/950,598, filed Sep. 13, 2001, now U.S. Pat. No. 6,716,234. This application claims the benefit of U.S. provisional application Ser. No. 60/492,972, filed Aug. 7, 2003. The disclosure of each of these applications is hereby incorporated by reference.

### BACKGROUND OF THE INVENTION

1. Field of the Invention

The present invention relates to high strength surgical suture materials, and more particularly to braided suture blends of long-chain, ultrahigh molecular weight polyethylene and polyester that include collagen strands for improved tissue remodeling and biocompatibility.

2. Description of the Related Art

Suture strength and tissue compatibility are important considerations in any surgical suture material. One of the strongest materials currently formed into elongated strands is an ultrahigh molecular weight long chain polyethylene, typically used for fishing line and the like, which is sold under the trade names Dyneema or Spectra. This material is much stronger than ordinary surgical suture, however, it does not have acceptable knot tie down characteristics for use in surgical applications, and is not bioabsorbable.

Preparation of collagen fibers for use as surgical suture is known in the prior art. See U.S. Pat. Nos. 5,814,328 and 6,548,077 to Gunasekaran, U.S. Pat. No. 5,374,539 to Nimni et al., and U.S. Pat. No. 5,618,312 to Yui et al., for example.

A suture made of a high-strength blend of long-chain polyethylene and polyester developed by the assignee of the present application has acceptable tie down characteristics. See U.S. Pat. No. 6,716,234, the disclosure of which is hereby incorporated by reference. It would be advantageous to have high-strength suture with improved tissue compatibility and enhanced tissue remodeling capabilities.

### BRIEF SUMMARY OF THE INVENTION

The present invention advantageously provides a high strength surgical suture material with improved tissue compatibility. The suture features a braided cover made of a blend of ultrahigh molecular weight long chain polyethylene fiber and a fiber of one or more long chain synthetic polymers, preferably polyester. Fibers of collagen also are included in the braided cover. The polyethylene provides strength. The polyester improves tie down properties. The collagen enhances tissue remodeling and biocompatibility. Additionally, the collagen can form the base for adding growth factors to the suture, such as PDGF (platelet derived growth factor).

The preferred composition of the braided cover includes about 50% long-chain polyethylene, about 30% polyester, and about 20% collagen, preferably Type I bovine collagen. Filaments of Type I bovine collagen are provided by Kensey Nash Corporation. Preferably, about fifteen collagen filaments are braided together into ends. The ends are provided on bobbins, and are braided with the polyethylene and polyester into the braided suture cover.

Handling properties of the high strength suture, such as flexibility, hand, chatter, and drag, also can be enhanced using various materials to coat the suture. In addition, strands of a contrasting color can be added to the braided threads to enhance visibility. The colored strands preferably are dyed filaments of polyester or nylon. In an exemplary embodiment, half of a length of suture is provided with colored tracing strands, or otherwise contrasts with the other half of the length of suture, which remains a plain, solid color, for example. Accordingly, when the length of suture is loaded through the eyelet of a suture anchor, for example, identification and movement of the suture legs is simplified. The half-trace/half-plain arrangement of the suture facilitates tracing and identification of the two legs of the suture during arthroscopic surgery.

In a preferred embodiment, the suture includes a multifilament cover formed of ultrahigh molecular weight polyethylene fiber braided with polyester and/or nylon fibers, and Type I bovine collagen fibers. The cover surrounds a fiber core made substantially or entirely of ultrahigh molecular weight polyethylene. The core preferably includes three strands of ultrahigh molecular weight polyethylene, twisted at about three to six twists per inch.

The braided cover preferably includes eight strands of ultrahigh molecular weight polyethylene braided with six strands of polyester and two Type I bovine collagen strands. In addition, strands of nylon can be provided in black or some other contrasting color as described in co-pending U.S. application Ser. No. 10/358,399 incorporated herein by reference.

Ultrahigh molecular weight polyethylene fibers suitable for use in the present invention are marketed under the Dyneema trademark by Toyo Boseki Kabushiki Kaisha, and are produced in the U.S. by Honeywell International Inc., under the trademark Spectra.

The suture of the present invention advantageously has the strength of Ethibond #5 suture (Ethicon, Inc.), yet has the diameter, feel and tie ability of #2 suture, and promotes remodeling of surrounding tissue. As a result, the suture of the present invention is ideal for most orthopedic procedures such as rotator cuff repair, Achilles tendon repair, patellar tendon repair, ACL/PCL reconstruction, hip and shoulder reconstruction procedures, and replacement for suture used in or with suture anchors.

The suture can be uncoated, or coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

As an added advantage, as mentioned above, all or some of the nylon fibers (or the polyester fibers) in the cover may be provided in a contrasting color for visibility and identification purposes. A few trace threads having a contrasting color, preferably of a readily dyed fiber such as polyester or nylon, in the cover aid surgeons in identifying the travel direction of the suture during surgery, particularly during arthroscopic operations. Providing the trace threads in a regularly repeating pattern is particularly useful, allowing the surgeon to distinguish different ends of lengths of suture, and determine the direction of travel of a moving length of suture. The trace threads preferably are provided on only half of a length of suture to allow for tracing and identification of the two halves of the suture, such as when the suture is threaded through an eyelet of a suture anchor. Of the more easily dyed fibers, nylon is preferred in that it accepts dye readily. Polyester fibers are stronger, but do not take up dye as easily as nylon.

The present invention is not limited by the type of collagen fiber used or the method by which is it obtained. Collagen fibers used in the present invention can be produced, extracted, or harvested from animal tissue, and may be generated or modified by various processes, including enzymatic treatment of collagen material also including treatments using microorganisms. The result collagen products or intermediates can undergo separation and purification, and may be enzymatically or chemically modified to enhance various properties of the collagen-based material. It may also be possible to derive collagen fibers by way of synthetic methods for manufacturing polymer proteins or polypeptides. Moreover, as mentioned previously, growth factors, such as PDGF, can be added to the collagen strands.

The suture, including the cover and the core, and the strands making up the suture, can be formed from individual or multiple filaments or yarns by various known methods, including but not limited to being entangled, twisted, plied, braided, and knitted.

Other features and advantages of the present invention will become apparent from the following description of the invention which refers to the accompanying drawings.

### BRIEF DESCRIPTION OF THE DRAWINGS

FIG. 1 is a copy of a scanning electron micrograph of a length of suture according to the present invention.

FIG. 2 is a schematic cross section of a length of suture according to the present invention.

FIG. 3 is an illustration of the suture of the present invention attached to a suture anchor.

FIGS. 4A and 4B show the suture of the present invention attached to a half round, tapered needle.

FIG. 5 illustrates a bulk length of suture of the present invention.

FIG. 6 illustrates a strand of suture according to the present invention provided on a suture anchor.

### DETAILED DESCRIPTION OF THE INVENTION

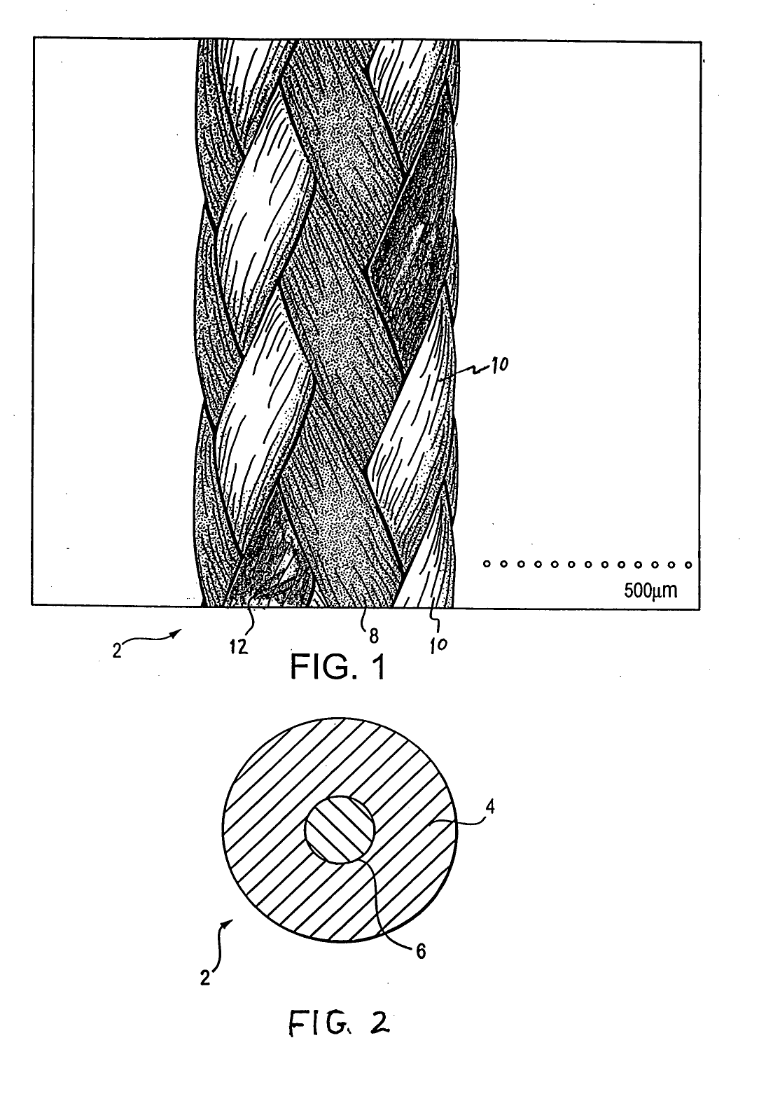

Referring to FIG. 1, a scanning electron micrograph of a length of suture 2 according to the present invention is shown. Suture 2 is made up of a cover 4 and a core 6 surrounded by the cover. See FIG. 2. Strands of long-chain, ultrahigh molecular weight polyethylene (UHMWPE) 8, sold under the tradename Spectra or Dyneema, strands of polyester 10 and strands of collagen 12 are braided together to form the cover 4. Core 6 is formed of twisted strands of UHMWPE.

UHMWPE strands 8 are substantially translucent or colorless. The majority of the polyester strands 10 are white (undyed). Some of the polyester or nylon strands 10 having a contrasting color can be included to provide a trace in the suture. Due to the transparent nature of the UHMWPE, the suture takes on the color of strands 10, and thus appears to be white, with a trace in the contrasting color.

Details of the present invention will be described further below in connection with the following examples:

Example: USP Size 5 (EP size 7)

Made on a 16 carrier Hobourns machine, the yarns used in the braided cover are Honeywell Spectra 2000, polyester type 712, and Type I collagen. The cover is formed using eight strands of 144 decitex Spectra per carrier, braided with six strands of 100 decitex polyester, and two strands of collagen. The core is formed of three carriers of 144 decitex Spectra braided at three to six twists per inch. Alternatively, collagen fibers can be incorporated into the core as well.

The example set forth above is for size 5 suture. In the making of various sizes of the inventive suture, different decitex values and different PPI settings can be used to achieve the required size and strength needed. In addition, smaller sizes may require manufacture on 12 carrier machines, for example. The very smallest sizes preferably are made without a core. Overall, the suture may range from 5% to 90% ultrahigh molecular weight polymer (preferably at least 40% of the fibers are ultrahigh molecular weight polymer), with the balance formed of polyester and/or nylon, and collagen. Collagen preferably is present in amounts of about 20% of the braided cover. The core preferably comprises 18% or greater of the total amount of filament.

The suture preferably is coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

The ultra high molecular weight (UHMW) polymer component of the present invention provides strength, and the polyester component is provided to improve tie ability and tie down characteristics. However, it has been found that the UHMW polymer provides an unexpected advantage of acting as a cushion for the polyester fibers, which are relatively hard and tend to damage each other. The UHMW polymer prevents breakage by reducing damage to the polyester when the suture is subjected to stress.

According to an alternative embodiment of the present invention, bioabsorbable suture is provided by blending a high strength material, such as UHMWPE fibers, with a bioabsorbable material, such as PLLA or one of the other polylactides, for example, in addition to the collagen. Accordingly, a suture made with about 10% Spectra or Dyneema blended with absorbable fibers would provide greater strength than existing bioabsorbable suture with less stretch. Over time, 90% or more of the suture would absorb, leaving only a very small remnant of the knot. The absorbable suture can include coatings and colored traces as noted above for suture containing collagen as the only absorbable component.

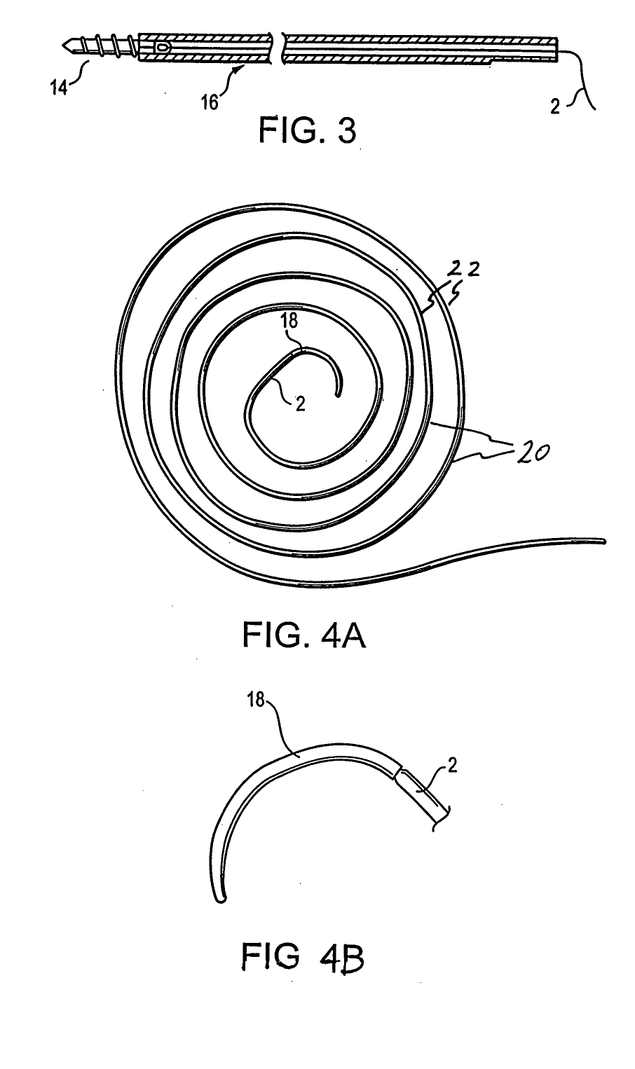

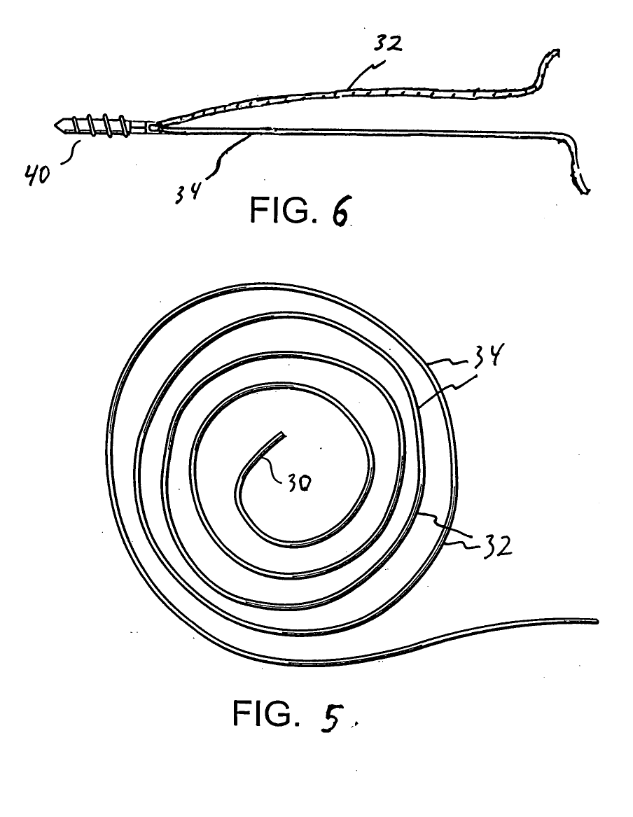

In one method of using the suture of the present invention, the suture 2 is attached to a suture anchor 14 as shown in FIG. 3 (prepackaged sterile with an inserter 16), or is attached at one or both ends to a half round, tapered needle 18 as shown in FIGS. 4A and 4B. FIG. 4A also illustrates a length of suture having regularly repeating pattern of trace threads according to the present invention. Sections 20 of the length of suture 2 have tracing threads woven in, where sections 22 of the length of suture are plain, or otherwise are distinguishable from sections 20. The alternating patterned and plain sections aid the surgeon in determining the direction of suture travel, for example.

As shown in FIG. 5, bulk suture 30 is provided with repeating sections 32 having trace threads separated by sections 34 having no trace threads. The bulk suture is cut between every other section, at one end of each plain section, for example, to provide lengths of suture that are half traced and half plain. Alternatively, the bulk suture can be cut midway through each section to provide a shorter suture having a trace at one end. The half-and-half lengths of suture can be threaded through the eyelet of a suture anchor 40, as shown in FIG. 6. Accordingly, each leg of the suture strand provided on the suture anchor is easily distinguishable by a surgeon operating with the suture anchor assembly.

By providing strands of collagen, or other bioabsorbable polypeptides, in the suture, tissue remodeling is enhanced. Accordingly, as the collagen or other polpyeptide degrades, it is replaced by newly formed tissue generated by that surrounding the suture. As a result, remodeling of the tissue surrounding the repair site is enhanced. Additionally, the collagen strands can serve as a base for the addition of growth factor, such as PDGF, to the suture.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

1. A suture strand suitable for use as a suture or ligature comprising a plurality of braided fibers of ultrahigh molecular weight polyethylene, a plurality of fibers of at least one long chain synthetic polymer or bioabsorbable fiber, and a plurality of fibers of a collagen, the fibers being woven to produce the suture strand.

2. A suture strand as recited in claim 1, wherein sections of at least one of the plurality of fibers is provided in a color contrasting from the other fibers to provide an identifiable trace along a portion of the suture strand.

3. A suture strand as recited in claim 2, wherein at least one portion of the suture strand is provided with no identifiable trace.

4. A suture strand as recited in claim 1, wherein the at least one long chain synthetic polymer is polyester, nylon, or both.

5. A suture strand as recited in claim 1, wherein the ultrahigh molecular weight polyethylene comprises at least 40% of the braided fibers.

6. A suture strand as recited in claim 1, wherein the polyester comprises less than about 40% of the braided filaments.

7. A suture strand as recited in claim 1, wherein the collagen comprises about 20% of the braided filaments.

8. A suture strand as recited in claim 1, further comprising a core of twisted fibers of ultrahigh molecular weight polyethylene surrounded by a cover comprising the plurality of braided fibers of ultrahigh molecular weight polyethylene, synthetic fiber, and collagen.

9. A suture strand as recited in claim 8, wherein the core comprises about 18% or greater of the total amount of filament in the strand.

10. A suture strand as recited in claim 8, wherein the core further includes fibers of collagen.

11. A suture strand as recited in claim 8, wherein the cover comprises less than about 82% of the total amount of filament in the strand.

12. A suture strand as recited in claim 8, further comprising a coating disposed on the cover.

13. A suture strand as recited in claim 12, wherein the coating is selected from the group consisting of wax, silicone, silicone rubbers, PTFE, PBA, and ethyl cellulose.

14. A suture strand as recited in claim 1, wherein a half length of the strand is provided with an identifiable trace, and the other half length of the strand contains no identifiable trace.

15. A suture assembly comprising:
a suture comprising fibers of ultrahigh molecular weight polyethylene; and
a suture anchor attached to the suture.

16. A suture assembly as recited in claim 15, wherein the suture further comprises fibers of polyester, nylon, bioabsorbable material or a combination thereof, which are braided with the fibers of ultrahigh molecular weight polyethylene.

17. A suture assembly as recited in claim 15, wherein the suture further comprises fibers of collagen which are braided with the fibers of ultrahigh molecular weight polyethylene.

18. A suture assembly as recited in claim 16, wherein the suture further comprises fibers of collagen which are braided with the fibers of ultrahigh molecular weight polyethylene and the fibers of polyester, nylon, bioabsorbable material or a combination thereof.

19. A suture assembly as in claim 16, wherein the fibers of polyester, nylon or bioabsorbable material are provided in a color contrasting with the remaining fibers on substantially one half length of the suture, and a core formed of twisted fibers of ultrahigh molecular weight polyethylene.

20. A suture assembly comprising:
a suture comprising fibers of ultrahigh molecular weight polyethylene; and
a half round, tapered needle attached to one or both ends of the suture.

21. A suture assembly as recited in claim 20, wherein the suture further comprises fibers of polyester, nylon, bioabsorbable material or a combination thereof, which are braided with the fibers of ultrahigh molecular weight polyethylene.

22. A suture assembly as recited in claim 20, wherein the suture further comprises fibers of collagen which are braided with the fibers of ultrahigh molecular weight polyethylene.

23. A suture assembly as recited in claim 21, wherein the suture further comprises fibers of collagen which are braided with the fibers of ultrahigh molecular weight polyethylene and the fibers of polyester, nylon, bioabsorbable material or a combination thereof.

24. A suture assembly as in claim 21, wherein the fibers of polyester, nylon or bioabsorbable material are provided in a color contrasting with the remaining fibers on substantially one half length of the suture, and wherein the suture further comprises a core formed of twisted fibers of ultrahigh molecular weight polyethylene.

25. A suture strand suitable for use as a suture or ligature having at least an outer cover comprising a plurality of braided fibers including synthetic fibers and collagen fibers.

26. The suture strand of claim 25, further comprising a coating selected from the group consisting of wax, silicone, silicone rubbers, PTFE, PBA, and ethyl cellulose.

27. A method of making suture comprising the steps of:
forming a braided end of long-chain polyethylene;
forming a braided end of a fiber selected from the group consisting of polyester and nylon;
forming a braided end of a collagen fiber; and
braiding the braided ends together to form a braided cover.

28. A method of making suture according to claim 27, wherein the braided cover is formed over a filamentous core.

29. A method of making suture according to claim 28, wherein the filamentous core includes fibers of collagen.
